(19) United States
(12) Patent Application Publication
(10) Pub. No.: US 2024/0245913 A1
(43) Pub. Date: Jul. 25, 2024

(54) Hearing Device, Hearing Device-Based Systems and Methods for Monitoring Glucose

(71) Applicant: SONOVA AG, Staefa (CH)

(72) Inventor: Maryam Mostafaei, Wallisellen (CH)

(21) Appl. No.: 18/099,699

(22) Filed: Jan. 20, 2023

(51) Int. Cl. A61N 1/36; A61B 5/145

(52) U.S. Cl. CPC A61N 1/36038; A61B 5/145

(57) ABSTRACT

An illustrative hearing system may include a housing having at least one light source and at least one detector. The at least one light source may be configured to emit a first light having a first excitation frequency and a second light having a second excitation frequency toward tissue at the ear. The at least one detector may be configured to detect intensities of the first light at frequencies offset from the first excitation frequency after the first light has been scattered by the tissue and intensities of the second light at frequencies offset from the second excitation frequency after the second light has been scattered by the tissue. A processing unit may be configured to determine, based on the intensities of the first light and the second light, a glucose value representative of a glucose concentration within the tissue.

### BACKGROUND INFORMATION

A hearing device may enable or enhance hearing by a user wearing the hearing device by providing audio content received by the hearing device to the user. For example, a hearing aid may provide an amplified version of the audio content in the form of environmental sound at the location of the user to the user to enhance hearing by the user. A hearing device may further provide audio content to the user based on an audio stream (e.g., an electrical audio signal) received by the hearing device from a streaming source (e.g. a table microphone or a streaming service). As another example, a hearing device may include a cochlear implant system and a sound processor included in the cochlear implant system may provide electrical stimulation representative of the audio content to the user to enable hearing by the user.

In some cases, it may be desirable to monitor a blood sugar or glucose level of a user (e.g., to manage diabetes). In some scenarios, a glucose level may be determined by obtaining a blood sample from the user and measuring the glucose level in the blood sample. Unfortunately, obtaining a blood sample from the user may involve pricking the user (e.g., at a fingertip), which may be painful and/or cause anxiety for the user.

### BRIEF DESCRIPTION OF THE DRAWINGS

The accompanying drawings illustrate various embodiments and are a part of the specification. The illustrated embodiments are merely examples and do not limit the scope of the disclosure. Throughout the drawings, identical or similar reference numbers designate identical or similar elements.

FIG. 1 shows an illustrative implementation of a hearing system.

FIG. 2 shows another illustrative implementation of a hearing system.

FIG. 3A shows an illustrative implementation of a hearing device.

FIG. 3B shows another illustrative implementation of a hearing device.

FIG. 4 shows an illustrative method of operating a hearing system.

FIG. 5 shows an illustrative implementation of operating a hearing system.

FIG. 6A shows an illustrative implementation of spectra that may be identified by a hearing system.

FIG. 6B shows another illustrative implementation of spectra that may be identified by a hearing system.

FIG. 7 shows another illustrative implementation of operating a hearing system.

FIG. 8 shows another illustrative method of operating a hearing system.

FIG. 9 shows an illustrative computing system according to the principles described herein.

### DETAILED DESCRIPTION

An illustrative hearing system may be configured to monitor glucose levels (e.g., blood glucose levels) within tissue (e.g., skin, blood, muscles, etc.) at an ear of a user. For example, an illustrative hearing system may be positioned at the ear of the user (e.g., within the ear canal, behind the ear, etc.) and configured to emit light toward tissue at the ear. As the light is scattered by the tissue, the light may interact with molecules within the tissue. In some instances, this interaction may cause the frequency of the light to shift such that the light scattered by the tissue may have a different frequency than the light emitted toward the tissue by the hearing system. The scattered light may be detected by a detector of the hearing system and used to determine a glucose level within the tissue.

For example, an illustrative hearing system may include a housing configured to be worn at an ear of a user. The housing may include at least one light source configured to emit a first light and a second light toward tissue at the ear. The first light may have a first excitation frequency and the second light may have a second excitation frequency different from the first excitation frequency. The housing may further include at least one detector configured to detect intensities of the first light at frequencies offset from the first excitation frequency after the first light has been scattered by the tissue and intensities of the second light at a frequencies offset from the second excitation frequency after the second light has been scattered by the tissue. A processing unit may be configured to determine, based on the intensities of the first light and the second light, a glucose value representative of a glucose concentration within the tissue.

To illustrate, glucose molecules within the tissue may affect the intensities of the first light and/or second light at one or more predetermined frequencies offset from the first excitation frequency and/or the second excitation frequency such that the intensities of the first light and/or the second light at the one or more predetermined frequencies may be correlated to the glucose value. In some instances, a change in the intensities of the first light and/or second light at the one or more predetermined frequencies may indicate a change in the glucose concentration within the tissue.

In some implementations, the first light may penetrate the tissue at a first depth and the second light may penetrate the tissue at a second depth greater than the first depth such that the determining the glucose value may include determining the intensities of the first light that was scattered by the tissue at the first depth and determining the intensities of the second light that was scattered by the tissue at the second depth.

In some implementations, the hearing system may detect the intensities of the first light and the second light at a single location from the at least one light source. Alternatively, the hearing system may detect the intensities of the first light at a first distance from the at least one light source and the intensities of the second light at a second distance from the at least one light source greater than the first distance.

In some implementations, the housing may be included in a hearing device configured to be at least partially positioned within an ear canal and/or behind the ear of a user. The hearing device may further include a microphone configured to detect an audio signal presented to the user and an output transducer configured to output the audio signal detected by the microphone to the user.

The principles described herein may result in improved hearing systems compared to conventional devices that do not include detecting intensities of light at shifted frequencies to determine a glucose value, as well as provide other benefits as described herein. For example, a hearing system that detects intensities of light at shifted frequencies to determine a glucose value may provide a non-invasive approach (e.g., without obtaining a blood sample) for monitoring glucose levels within the tissue, such as blood glucose levels. This non-invasive approach may decrease the pain and/or anxiety associated with measuring glucose levels (e.g., from finger-pricking), which may allow a user to perform more regular, quicker, and/or efficient glucose measurements. Moreover, the determination of glucose levels using a wearable device at least partially positioned at the ear of the user may further improve long-term monitoring of glucose. For example, the wearable device may be in contact with tissue at the ear for an extended period of time. These benefits may provide better management of diabetes and/or save medical costs for the user.

Various embodiments will now be described in more detail with reference to the figures. The systems, hearing devices, and methods described herein may provide one or more of the benefits mentioned above and/or various additional and/or alternative benefits that will be made apparent herein. While monitoring glucose levels within tissue, such as blood glucose levels, is described herein, the systems, hearing devices, and methods may additionally or alternatively be configured to monitor other biological compounds (e.g., lipids, proteins, amino acids, carbohydrates, hemoglobin, volatile organic compounds, chemical elements, hormones, antibodies, enzymes, etc.) and/or properties (e.g., an oxygen saturation index (SpO₂), blood pressure, heart rate, etc.) within the tissue.

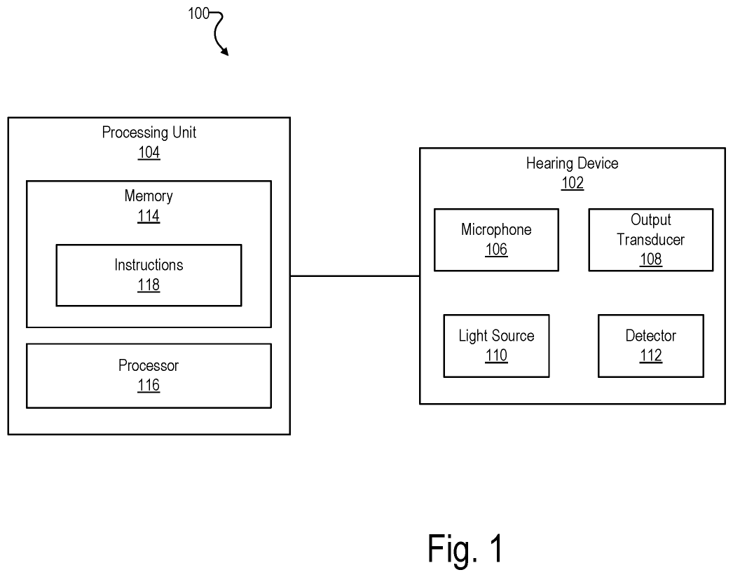

FIG. 1 shows an illustrative implementation 100 of a hearing system configured to monitor glucose within tissue at the ear. As shown, implementation 100 includes a hearing device 102 communicatively coupled with a processing unit 104. Implementation 100 may include additional or alternative components as may serve a particular implementation.

Hearing device 102 herein is to be understood as any device configured to output sound to a user that includes components that are worn at least partially in an ear canal of a user. In particular, hearing device 102 may be implemented as a hearing aid or a hearing instrument configured to enable or enhance hearing by a user wearing hearing device 102. For example, hearing device 102 may be implemented by a hearing aid configured to provide an amplified version of audio content to a user, a sound processor included in a cochlear implant system configured to provide electrical stimulation representative of audio content to a user, a sound processor included in a cochlear implant system configured to provide electrical stimulation representative of audio content to a user, a sound processor included in a bimodal hearing system configured to provide both amplification and electrical stimulation representative of audio content to a user, or any other suitable hearing prosthesis. As described herein, hearing device 102 may include one or more components configured to be at least partially positioned within an ear canal of the user. For example, hearing device 102 may be implemented by earbuds, earphones, hearables, and/or hearing instruments such as a Receiver-In-Canal (RIC) device, an In-The-Ear (ITE) device, a Completely-In-Canal (CIC) device, and/or an Invisible-In-The-Canal (IIC) device. Additionally or alternatively, hearing device 102 may be implemented by a behind-the-ear (BTE) device configured to be worn behind an ear of a user.

As shown, hearing device 102 includes a microphone 106, an output transducer 108, at least one light source 110, and at least one detector 112. Hearing device 102 may include additional or alternative components as may serve a particular implementation. Such additional or alternative components may comprise, for example, at least one of a battery, a power management system, a communication interface (e.g., a Bluetooth radio), and/or a user interface (e.g., a push button) to control the hearing device 102.

Microphone 106 may be implemented by one or more suitable audio detection devices configured to detect an audio signal presented to a user of hearing device 102. In particular, microphone 106 may include an electro acoustic transducer configured to convert an audio signal in the form of an acoustic signal, e.g. sound, into an electric audio signal, i.e. an electric signal comprising information representative of the acoustic signal. The audio signal may include, for example, audio content (e.g., music, speech, noise, etc.) generated by one or more audio sources included in an environment of the user. Microphone 106 may be included in or communicatively coupled to hearing device 102 in any suitable manner. In some implementations, an interface may be included in addition to or instead of microphone 106 to receive an audio stream such as an electrical audio signal from a streaming source (e.g., a table microphone and/or a streaming service). Output transducer 108 may be implemented by any suitable audio output device, for instance a loudspeaker, also called ‘receiver’, of a hearing device or an output electrode of a cochlear implant system.

Light source 110 may be implemented by any suitable device (e.g., a light-emitting diode (LED), a laser diode, etc.) configured to emit light. For example, light source 110 may be configured to emit light toward tissue at an ear of a user. In some implementations, light source 110 may emit light in a near-infrared (NIR) range (e.g., light having a wavelength between about 750 nanometers and about 2,500 nanometers, such as about 780 nanometers to about 1,000 nanometers, such as about 785 nanometers to about 830 nanometers) and/or other at other suitable wavelengths that may be scattered by tissue at the ear. For example, light source 110 may include a broad band light source (e.g., an infrared transmitter, etc.) and a band pass filter for emitting light in the NIR range. In some implementations, light source 110 may further be configured to emit monochromatic light. Light source 110 may include one or more light sources. In some implementations, light source 110 may include a plurality of light sources that may be configured to emit light at substantially the same wavelengths and/or different wavelengths.

Detector 112 may be implemented by any suitable device configured to detect light. For example, detector 112 may be configured to detect light emitted by light source 110 after the light has been scatter by tissue at the ear. In some implementations, detector 112 may detect light in the NIR range. To illustrate, detector 112 may include a photodetector, a charge-coupled device (CCD) a mercury cadmium telluride (MCT) detector, Indium Gallium Arsenide (InGaAs), and/or other type of infrared receiver. In some implementations, detector 112 may include a single detector. Additionally or alternatively, detector 112 may include a plurality of detectors that may be configured to detect light at substantially the same wavelengths and/or different wavelengths. In some instances, at least one lens is positioned in front of detector 112, e.g., to increase the detection efficiency.

Processing unit 104 may be implemented by one or more computing devices and/or computer resources (e.g., processors, memory devices, storage devices, etc.) as may serve a particular implementation. For example, processing unit 104 may be implemented by a mobile device, personal computer, and/or other computing device configured to be communicatively coupled (e.g., by way of a wired and/or wireless connection) to hearing device 102. As shown, processing unit 104 may include, without limitation, a memory 114 and a processor 116 selectively and communicatively coupled to one another. Memory 114 and processor 116 may each include or be implemented by computer hardware that is configured to store and/or process computer software. Various other components of computer hardware and/or software not explicitly shown in FIG. 1 may also be included within processing unit 104. In some examples, memory 114 and/or processor 116 may be distributed between multiple devices and/or multiple locations as may serve a particular implementation.

Memory 114 may store and/or otherwise maintain executable data used by processor 116 to perform any of the functionality described herein. For example, memory 114 may store instructions 118 that may be executed by processor 116. Memory 114 may be implemented by one or more memory or storage devices, including any memory or storage devices described herein, that are configured to store data in a transitory or non-transitory manner. Instructions 118 may be executed by processor 116 to cause processing unit 104 to perform any of the functionality described herein. Instructions 118 may be implemented by any suitable application, software, code, and/or other executable data instance. Additionally, memory 114 may also maintain any other data accessed, managed, used, and/or transmitted by processor 116 in a particular implementation.

Processor 116 may be implemented by one or more computer processing devices, including general purpose processors (e.g., central processing units (CPUs), graphics processing units (GPUs), microprocessors, etc.), special purpose processors (e.g., application specific integrated circuits (ASICs), field-programmable gate arrays (FPGAs), etc.), image signal processors, or the like. Using processor 116 (e.g., when processor 116 is directed to perform operations represented by instructions 118 stored in memory 114), processor 116 may perform various operations as described herein.

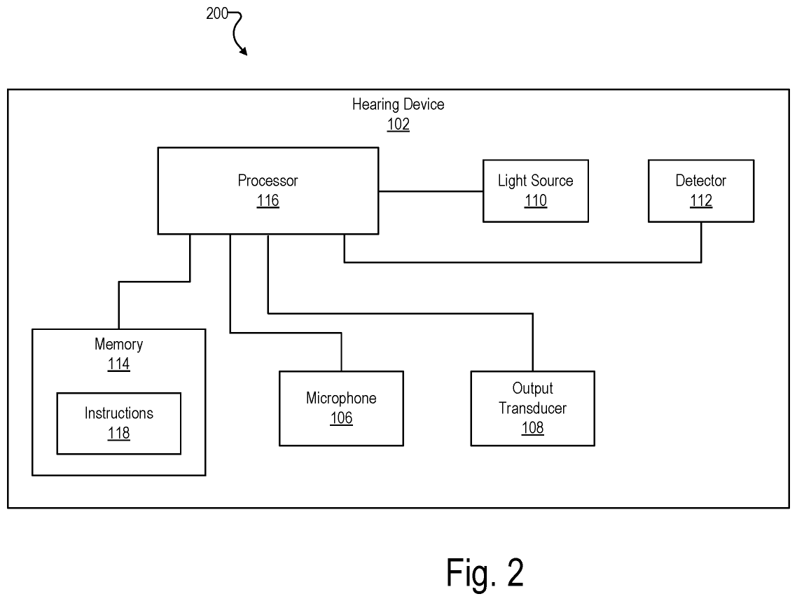

FIG. 2 shows another illustrative implementation 200 of a hearing system configured to monitor glucose within tissue at the ear. As shown, implementation 200 is similar to implementation 100, except that implementation 200 includes memory 114 and processor 116 located within hearing device 102. Implementation 200 may include additional or alternative components as may serve a particular implementation.

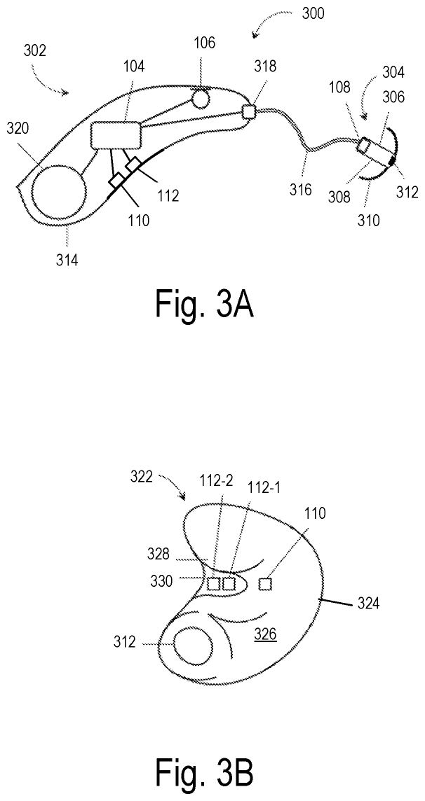

FIG. 3A shows an illustrative implementation 300 of hearing device 102 as a RIC device. As shown, RIC device 300 comprises a BTE portion 302 configured to be worn at an ear at a wearing position behind the ear, and an ITE portion 304 configured to be worn at the ear at a wearing position at least partially inside an ear canal of the ear. ITE portion 304 comprises a first housing 306 including a body portion 308 and a sealing member 310 coupled to body portion 308. Body portion 308 is configured to enclose output transducer 108. Sealing member 310 is adapted to contact an ear canal wall when first housing 306 is at least partially inserted into the ear canal to at least partially block ambient sound from entering the ear canal. Sealing member 310 may be implemented, for instance, as a flexible member adaptable to the ear canal shape, which may have a shape of a dome, or a shell having a shape customized to the shape of an individual ear canal. A sound outlet 312 may be implemented as an opening at body portion 308 and/or at sealing member 310.

BTE portion 302 of RIC device 300 comprises a second housing 314 configured to be worn behind the ear. Second housing 314 accommodates processing unit 104 communicatively coupled to microphone 106, light source 110, and detector 112. As shown, light source 110 and detector 112 are positioned at an exterior surface of second housing 314 such that light source 110 may be configured to emit light toward tissue at the ear (e.g., behind the ear) and detector 112 may be configured to detect light after the light has been scattered by the tissue. In some implementations, detector 112 is spaced a select distance away from light source 110. Processing unit 104 is communicatively coupled to output transducer 108 by a cable 316 and a cable connector 318 provided at second housing 314. BTE portion 302 may further include a battery 320 as a power source for the above-described components.

FIG. 3B shows another illustrative implementation 322 of hearing device 102 that includes a housing 324 at least partially insertable into an ear canal. Housing 324 is provided as a shell having a shape that may be customized to the shape of an individual ear canal. A surface 326 of housing 324 may at least partially contact a wall of the ear canal when housing 324 is at least partially inserted into the ear canal to constitute a sealing member configured to form an acoustic seal with the ear canal wall at least partially blocking ambient sound from entering the ear canal. In the illustrated example, housing 324 includes a concave curvature 328 configured to conform to a protrusion of the ear canal wall, which may be formed at a bend of the ear canal wall. Sound outlet 312 is provided at an end of housing 324 facing an inner region of the ear canal when housing 324 is at least partially inserted into the ear canal.

Light source 110 and detectors 112 (e.g., detectors 112-1 to 112-2) may be provided at surface 326 of housing 324 and/or or accommodated inside housing 324. In the illustrated example, light source 110 and detectors 112 are provided at concave curvature 328 of housing 324, more particularly at a middle portion 330 of concave curvature 328 configured to contact the ear canal at a middle portion of the protrusion of the ear canal. Accordingly, when housing 324 is at least partially inserted into the ear canal, the light emitted from light source 110 may illuminate tissue at the ear at the protrusion of the ear canal, and light scattered by the tissue at the protrusion of the ear canal may be received by detectors 112. Still other positions of light source 110 and/or detectors 112 may be used relative to hearing device 102 to sufficiently emit light toward tissue at the ear of the user and/or detect light scattered by tissue at the ear of the user.

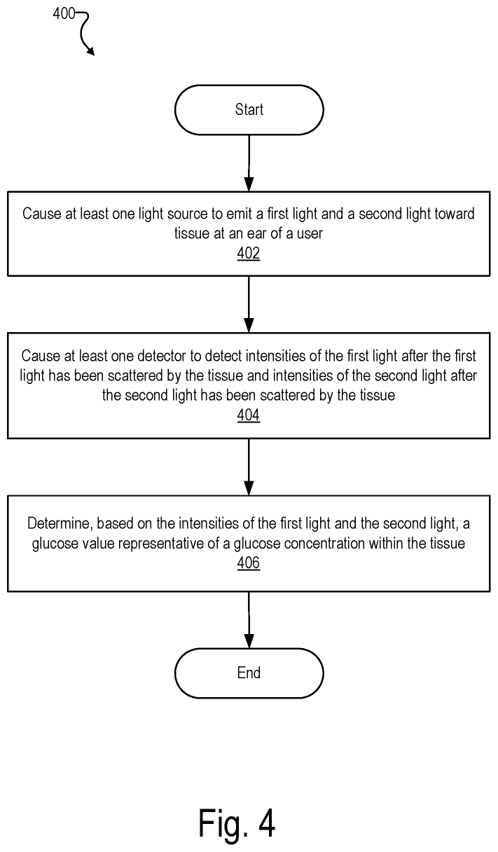

FIG. 4 shows an illustrative method 400 that may be performed by a hearing system according to the principles described herein. While FIG. 4 illustrates exemplary operations according to one embodiment, other embodiments may omit, add to, reorder, and/or modify any of the operations shown in FIG. 4. Moreover, each of the operations depicted in FIG. 4 may be performed in any of the ways described herein. In some implementations, each of the operations depicted in FIG. 4 may be performed by at least one computing device (e.g., by processor 116).

As shown, the hearing system may, at operation 402, cause at least one light source (e.g., light source 110) to emit a first light and a second light toward tissue at an ear of a user. In some implementations, the second light emitted by the at least one light source may have a different characteristic (e.g., a frequency, a wavelength, a wavenumber, etc.) than the first light emitted by the at least one light source. As an example, the first light may have a first excitation frequency and the second light may have a second excitation frequency different from the first excitation frequency (e.g., the second light may have a lower frequency than the first light). Additionally or alternatively, the second light may have a greater wavelength than the first light. In some instances, this may allow the second light to penetrate the tissue at a greater depth than the first light.

In some implementations, the first and second light emitted toward the tissue may be scattered by the tissue. For example, the emitted first and second light may interact with molecules within the tissue such that electrons within the molecules may absorb energy from photons of the emitted first and second light and transit to a higher energy state. As the emitted first and second light are scattered by the molecules, energy of the electrons within the molecules may decrease from the higher energy state and may scatter the photons of the emitted first and second light at frequencies offset from the first excitation frequency and/or the second excitation frequency. In some instances, the molecules may gain energy overall from the photons during the scattering, which may result in the scattered photons losing energy and having increased wavelengths (e.g., Stokes Raman scattering).

Hearing system may further, at operation 404, cause at least one detector (e.g., detector 112) to detect intensities of the first light after the first light has been scattered by the tissue and intensities of the second light after the second light has been scattered by the tissue. For example, the at least one detector may be configured to detect intensities of the first light at frequencies offset from the first excitation frequency after the first light has been scattered by the tissue and intensities of the second light at frequencies offset from the second excitation frequency after the second light has been scattered by the tissue.

In some implementations, the at least one detector may be configured to detect the intensities of the first light and the second light at a single location relative to the at least one light source. Alternatively, the at least one detector may be configured to detect the intensities of the first light at a first location relative to the at least one light source and the intensities of the second light at a second location relative to the at least one light source spaced a distance away from the first location. To illustrate, the second location may be positioned at a greater distance from the at least one light source than the first location.

Hearing system may further, at operation 406, determine, based on the intensities of the first light and the second light, a glucose value representative of a glucose concentration within the tissue. For example, glucose molecules within the tissue may affect the intensities of the first light and/or second light at one or more predetermined frequencies offset from the first excitation frequency and/or the second excitation frequency. Accordingly, the intensities of the first light and/or the second light may be correlated to the glucose concentration within the tissue at the one or more predetermined frequencies offset from the respective first excitation frequency and/or second excitation frequency.

The glucose value may be represented by any suitable metric, such as a discrete value (e.g., a concentration, a level, a range, a percentage, etc.) representative of a glucose concentration within the tissue. In some implementations, the glucose value may be a relative value which may be indicative, e.g., of the momentary glucose concentration relative to an earlier glucose concentration of the user. E.g., the glucose value determined from intensities of the first light and/or second light at a momentary time may be compared to a glucose value which has been determined from intensities of the first light and/or second light at an earlier time to determine a relative change of the glucose value between the earlier time and the momentary time. In some implementations, the glucose value may be an absolute value which may be indicative of the glucose concentration in absolute units, e.g., in millimols per liter (mmol/L) or in milligrams per deciliter (mg/dL). E.g., the glucose value determined from the intensities of the detected first light and/or second light may be calibrated, at an initial time, with respect to the absolute glucose value measured under equal circumstances such that, when detected at a later time, the calibrated glucose value determined from the intensities of the first light and/or the second light is representative of the absolute glucose concentration. For instance, the absolute glucose value may be determined under the equal circumstances by a finger prick measurement which can then be used to calibrate the glucose value determined from the intensities of the detected first light and/or second light may be calibrated at the initial time. In some implementations, the glucose value may be representative of the glucose concentration within the tissue at a point in time and/or representative of a change of the glucose concentration within the tissue over a period of time. For example, the determining the glucose value may include comparing the intensities of the first light and/or the second light at the one or more predetermined frequencies with reference data representative of predetermined glucose values for the intensities of the first light and/or the second light at the one or more predetermined frequencies. Such reference data may be based on previous levels of intensities of the first light and/or the second light by the user and/or other users. In some examples, the reference data may be stored by the hearing system (e.g., in memory 114). Additionally or alternatively, the glucose value may be based on a change in the intensities of the first light and/or the second light over a period of time, which may be indicative of a change of the glucose concentration within the tissue over the period of time.

In some implementations, the hearing system may be matched with regard to the requirements of a user of the hearing system. To illustrate, the hearing system may be matched to the user by determining a baseline glucose value for the user. In some implementations, the baseline glucose value may be associated with an event (e.g., the morning, the evening, after eating, after taking medication, etc.). Glucose values determined after the baseline glucose value may be compared to the baseline glucose value for each respective event, such that a relative change in the glucose value from the baseline glucose value may be determined. In some implementations, as described above, the baseline glucose value may be a relative value of the glucose concentration. In some implementations, as described above, the baseline glucose value may be an absolute value of the glucose concentration. In some implementations, the glucose value determined from the intensities of the detected first light and/or the second light may be monitored over time and compared to an upper and/or lower threshold. E.g., the upper and/or lower threshold can be indicative of a critical glucose value which may be harmful to the user. In some implementations, in a case the glucose value determined from the intensities of the detected first light and/or second light exceeds the upper threshold and/or falls below the lower threshold, a warning notification may be output to the user. In some implementations, as described above, the upper and/or lower threshold may be a relative value of the glucose concentration. In some implementations, as described above, the upper and/or lower threshold may be an absolute value of the glucose concentration.

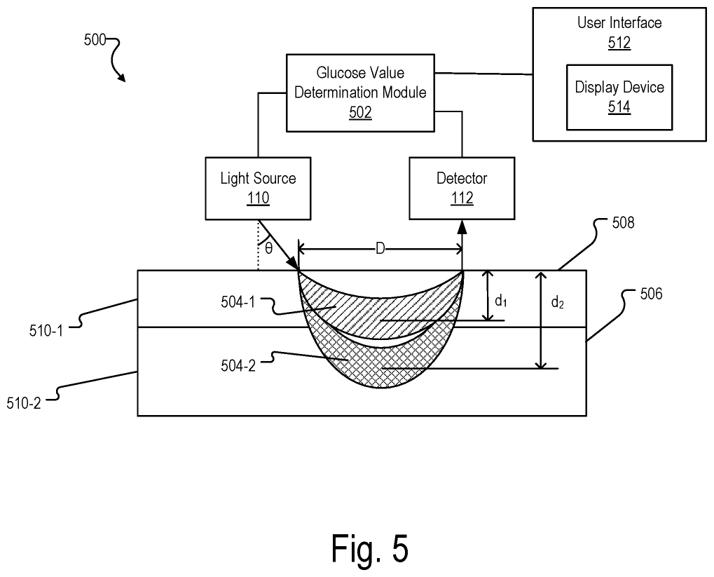

FIG. 5 shows an illustrative implementation 500 including a glucose value determination module 502 that may be implemented by a hearing system (e.g., by processor 116) according to the principles described herein and configured to determine a glucose value. Implementation 500 may include additional or alternative components as may serve a particular implementation.

As shown, glucose value determination module 502 may be configured to cause light source 110 to emit first light 504-1 and second light 504-2 toward tissue 506. First light 504-1 may have a first excitation frequency and second light 504-2 may have a second excitation frequency different from the first excitation frequency. In some implementations, the first excitation frequency may have a first wavelength (e.g., about 785 nanometers) and the second excitation frequency may have a second wavelength (e.g., about 830 nanometers) greater than the first wavelength. This may allow first light 504-1 to penetrate tissue 506 at a first depth d₁ (e.g., less than about 1 millimeter) and second light 504-2 to penetrate tissue 506 at a second depth d₂ (e.g., greater than about 1 millimeter, such as about 1 millimeter to about 10 millimeters) greater than first depth d₁ such that second light 504-2 may penetrate deeper into tissue 506 than first light 504-1.

In some instances, tissue 506 may comprise several layers 510 (e.g., layers 510-1 to 510-2) including at least one outer layer 510-1 and at least one inner layer 510-2. In such instances, first light 504-1 may be configured to penetrate tissue 506 at first depth d₁ within outer layer 510-1 and second light 504-2 may be configured to penetrate tissue 506 at second depth d₁ within inner layer 510-2. A transition between outer layer 510-1 and inner layer 510-2 may be gradual. In some instances, outer layer 510-1 and inner layer 510-2 may be provided near surface 508 of tissue 506 at such that outer layer 510-1 may comprise at least one skin layer and inner layer 510-2 may comprise at least one layer of subcutaneous tissue including blood vessels. In some implementations, the glucose value may be determined from intensities of the first light and/or second light after applying one or more conversion factors to the detected intensities. E.g., by applying the at least one conversion factor, a pure signal of the first light and/or the second light may be retrieved. In some instances, the pure signal may be defined as a signal indicative of the detected intensities of the light which has been only scattered at one of outer layer 510-1 and inner layer 510-2 of the tissue, in particular such that intensities corresponding to a fraction of the detected light which has been scattered at the other of outer layer 510-1 and inner layer 510-2 of the tissue is removed after applying the at least one conversion factor. E.g., by applying the at least one conversion factor to the detected intensities of the first light, the pure signal indicative of the detected intensities of the first light which has been only scattered at outer layer 510-1 may be retrieved. By applying the at least one conversion factor to the detected intensities of the second light, the pure signal indicative of the detected intensities of the second light which has been only scattered at inner layer 510-2 may be retrieved. The conversion factors can be predetermined for the first light and the second light detected by the at least one detector. E.g., at least one conversion factor may be associated with the first light and at least one conversion factor may be associated with the second light detected by the at least one detector.

In some implementations, glucose value determination module 502 may cause light source 110 to emit first light 504-1 and/or second light 504-2 at select intervals (e.g., about every 24 hours, about every 12 hours, about every 8 hours, about every 1 hour, about every 30 minutes, etc.) and/or based on a user input received from a user interface 512. For example, glucose value determination module 502 may be communicatively coupled (e.g., by way of a wired and/or wireless connection) with user interface 512 included in hearing device 102 and/or different wearable device separate from hearing device 102 (e.g., a watch or a mobile device worn or carried by the user) to receive a user input from user interface 512.

In some implementations, first light 504-1 and/or second light 504-2 may be emitted at an incident angle θ that is off-axis (e.g., less than about 90 degrees, such as about 40 degrees to about 80 degrees, such as about 60 degrees) relative to a normal of a surface 508 of tissue 506. In some implementations, light source 110 may include an optical waveguide extending between light source 110 and surface 508 of tissue 506 and configured to guide first light 504-1 and/or second light 504-2 toward tissue 506.

Glucose value determination module 502 may further be configured to cause detector 112 to detect intensities of first light 504-1 and second light 504-2 after first light 504-1 and second light 504-2 have been scattered by tissue 506. For example, detector 112 may be configured to detect the intensities of first light 504-1 at frequencies offset from the first excitation frequency and the intensities of second light 504-2 at frequencies offset from the second excitation frequency. In some implementations, glucose value determination module 502 may be configured to cause detector 112 to detect the intensities of first light 504-1 and second light 504-2 as first light 504-1 and second light 504-2 are scattered by tissue 506 (e.g., during a time period corresponding to the emitting of first light 504-1 and second light 504-2 by light source 110, such as up to about 30 seconds, up to about 10 seconds, and/or up to about 2 seconds).

In the illustrated implementation, detector 112 is configured to detect the intensities of first light 504-1 and second light 504-2 at a single location relative to light source 110. For example, the location may be positioned a select distance D (e.g., less than about 10 millimeters, such as about 1 millimeters to about 5 millimeters, such as about 3 millimeters) from a location where first light 504-1 and second light 504-2 contact surface 508 of tissue 506. In the illustrated implementation, detector 112 is configured to detect the intensities of first light 504-1 and second light 504-2 substantially perpendicularly relative to surface 508 of tissue 506, though any other suitable orientation may be used.

In some implementations, detector 112 may include an optical waveguide extending between detector 112 and surface 508 of tissue 506 that may be configured to guide first light 504-1 and/or second light 504-2 toward detector 112 after being scattered by tissue 506. Additionally or alternatively, detector 112 may include a filter (e.g., a notch filter, an edge pass filter, a band pass filter, etc.) configured to filter first light 504-1 at the first excitation frequency after first light 504-1 has been scattered by tissue 506 and/or second light 504-2 at the second excitation frequency after second light 504-2 has been scattered by tissue 506. This may allow detector 112 to detect the intensities of first light 504-1 at frequencies offset from the first excitation frequency and/or second light 504-2 at frequencies offset from the second excitation frequency more effectively.

Detector 112 may be configured to output detector data representative of the intensities of first light 504-1 and second light 504-2 at the frequencies detected by detector 112 to glucose value determination module 502. In some implementations, glucose value determine module 502 may be configured to process the detector data received by detector 112. For example, in some instances, the detector data may include the detection of first light 504-1 and second light 504-2 in a time-based domain such that glucose value determination module 502 may convert the detector data to a frequency-based domain (e.g., using a Fourier transform algorithm).

Glucose value determination module 502 may further be configured to determine, based on the intensities of first light 504-1 and second light 504-2, the glucose value representative of the glucose concentration within tissue 506. For example, glucose value determination module 502 may be configured to determine a parameter (e.g., a maximum, an average, a mean, a median, a mode, etc.) of the intensities of first light 504-1 and/or second light 504-2 at one or more predetermined frequencies offset from the respective first and second excitation frequencies. The predetermined frequencies may include frequencies at which glucose molecules within tissue 506 may affect the parameter of the intensities of first light 504-1 and/or second light 504-2. In some implementations, the parameter of the intensities of first light 504-1 and/or second light 504-2 at the one or more predetermined frequencies may be indicative of the glucose concentration within tissue 506 such that glucose value determination module 502 may correlate the parameter of the intensities of first light 504-1 and/or second light 504-2 at the one or more predetermined frequencies to the glucose value. In some implementations, glucose value determination module 502 may further be configured to output the glucose value to a display device 514 (e.g., included in user interface 512) to display the glucose value to the user.

In some implementations, the parameter of the intensities of first light 504-1 may be indicative of the glucose concentration within outer layer 510-1 of tissue 506 because first light 504-1 was scattered by tissue 506 at first depth d₁ within outer layer 510-1. Likewise, the parameter of the intensities of second light 504-2 may be indicative of the glucose concentration within inner layer 510-2 of tissue 506 because second light 504-2 was scattered by tissue 506 at second depth d₂ within inner layer 510-2. In some instances, inner layer 510-2 may include at least one layer of subcutaneous tissue having blood vessels such that the parameter of the intensities of second light 504-2 may correspond to a blood glucose concentration. Moreover, it may be desirable, in some instances, to determine the glucose value based on a blood glucose concentration such that the glucose value may be based on the parameter of the intensities of second light 504-2 scattered by tissue 506 at second depth d₂.

In some instances, the first light 504-1 scattered by tissue 506 may contribute to the intensities of light measured by detector 112 at second depth d₂ such that glucose value determine module 502 may be configured to differentiate intensities of light scattered by each layer 510 of tissue 506, such as by determining the intensities of light (e.g., first light 504-1) that was scattered by tissue 506 at first depth d₁ and the intensities of light (e.g., second light 504-2) that was scattered by tissue 506 at second depth d₂.

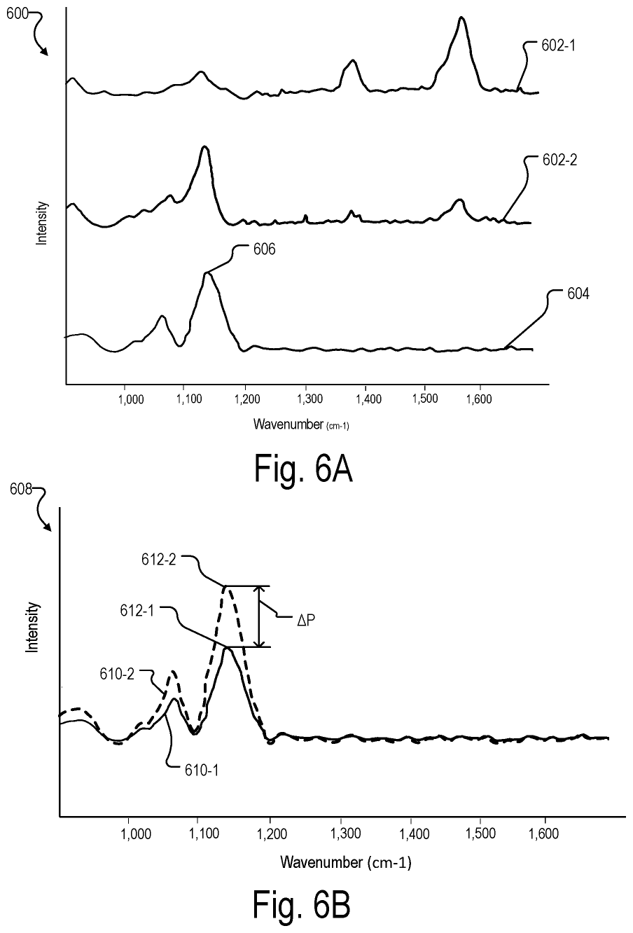

As an illustrative example, FIG. 6A shows an implementation 600 of spectra that may be identified by glucose value determine module 502. As shown, the spectra includes a first spectrum 602-1 representative of detected intensities of light at frequencies offset relative to the first excitation frequency and a second spectrum 602-2 representative of detected intensities of light at frequencies offset relative to the second excitation frequency. In the illustrated example, first spectrum 602-1 and second spectrum 602-2 are shown in terms of wavenumber (e.g., a Raman shift) and vertically spaced for illustrative purposes.

The detected intensities of first spectrum 602-1 may include contributions from light scattered by both outer layer 510-1 and inner layer 510-2 of tissue 506. Likewise, the detected frequencies of second spectrum 602-2 may include contributions from light scattered by both outer layer 510-1 and inner layer 510-2 of tissue 506. In some instances, a pure signal of first spectrum 602-1 may be retrieved which may only include the contributions from light scattered by outer layer 510-1 of tissue 506. In some instances, a pure signal of second spectrum 602-2 may be retrieved which may only include the contributions from light scattered by inner layer 510-2 of tissue 506. To retrieve the pure signal, the detected intensities of first spectrum 602-1 and/or second spectrum 602-2 may be converted by applying one or more conversion factors. For example, first spectrum 602-1 and second spectrum 602-2 may be represented by the equations below:

$${X}_{1} = {SF}_{1} \cdot {SS}_{1} + {SF}_{2} \cdot {SS}_{2} \\ {X}_{2} = {SF}_{3} \cdot {SS}_{1} + {SF}_{4} \cdot {SS}_{2}$$

where X₁ may represent the detected first spectrum 602-1, X₂ may represent the detected second spectrum 602-2, SS₁ may represent the pure signal of first spectrum 602-1, which may also be denoted as a first source spectrum including intensities of light scattered by outer layer 510-1 of tissue 506, SS₂ may represent the pure signal of second spectrum 602-2, which may also be denoted as a second source spectrum including intensities of light scattered by inner layer 510-2 of tissue 506, and SF (e.g., SF₁ to SF₄) may represent conversion factors relating the detected intensities X₁, X₂ of first and/or second spectrum 602-1, 602-2 to the pure signal SS₁, SS₂ of first and/or second spectrum 602-1, 602-2.

Glucose value determination module 502 may determine the pure signal SS₁ of first spectrum 602-1 and/or the pure signal SS₂ of second spectrum 602-2 by applying conversion factors SF to intensities X₁, X₂ of the detected first and/or second spectrum 602-1, 602-2, e.g., according to a predetermined mathematical operation and/or algorithm. For instance, glucose value determination module 502 may differentiate the source spectrum (e.g., SS₁ and SS₂) for each layer 510 of tissue 506, such as by applying the one or more conversion factors associated with the light detected by detector 112 (e.g., by a matrix transformation), as represented by the equations below:

$$[ \begin{matrix}X 1 \\ X 2\end{matrix} ] = [ \begin{matrix}SF 1 & SF 2 \\ SF 3 & SF 4\end{matrix} ] \cdot [ \begin{matrix}SS 1 \\ SS 2\end{matrix} ]$$

An example of a source spectrum 602-4 is shown in FIG. 6A that may be representative of intensities of light scattered by inner layer 510-2 of tissue 506 without contributions from light scattered by outer layer 510-2 of tissue 506. In the illustrated example, source spectrum 602-4 has been directly measured by a measurement technique only involving intensities of light scattered by inner layer 510-2 of tissue 506. However, source spectrum 602-4 may also be determined based on the detected first spectrum 602-1 and second spectrum 602-2 by applying conversion factors SF to intensities X₁, X₂ in the above described manner.

In some implementations, the intensities of source spectrum 604 at one or more predetermined frequencies may indicate the concentration of glucose within inner layer 510-2 of tissue 506. For example, vibrational modes for glucose molecules may be observed at predetermined frequency shifts (e.g., such as at wavenumbers about 800 cm⁻¹ to about 1200 cm⁻¹, such as about 911 cm⁻¹ to about 1125 cm⁻¹, such as about 1125 cm⁻¹) such that a peak 606 of the intensities of source spectrum 604 may be determined at the predetermined frequency shifts. In some implementations, peak 606 in the predetermined frequency shifts may be directly proportional to changes in glucose levels, while other peaks of the intensities of source spectrum due to solid tissue (e.g., lipid, protein, collagen, etc.) may remain unchanged.

For example, the glucose value may be determined based on a change in a peak intensity in the predetermined frequency shifts. As an illustrative example, FIG. 6B shows an implementation 608 including a first spectrum 610-1 and a second spectrum 610-2. First spectrum 610-1 may be acquired at a first time period and second spectrum 610-2 may be acquired at a second period after the first time period. First spectrum 610-1 and/or second spectrum 610-2 may include a spectrum representative of detected intensities (e.g., spectra 602) and/or the pure signal of the detected intensities.

In some implementations, a first peak 612-1 of first spectrum 610-1 and a second peak 612-2 of second spectrum 610-2 may be determined (e.g., at predetermined frequency shifts). A change ΔP between first peak 612-1 and second peak 612-2 may be determined, such as by subtracting first spectrum 610-1 and second spectrum 610-2 relative to each other. The change ΔP of peak intensity may be indicative of a change in the glucose concentration within the tissue between the first and second time periods. In some implementations, the change ΔP of peak intensity may be directly related to the change in glucose concentration such that the glucose value may increase as the change ΔP of peak intensity increases and/or decrease as the change ΔP of peak intensity decreases. Still other suitable techniques for determining the glucose value may be used.

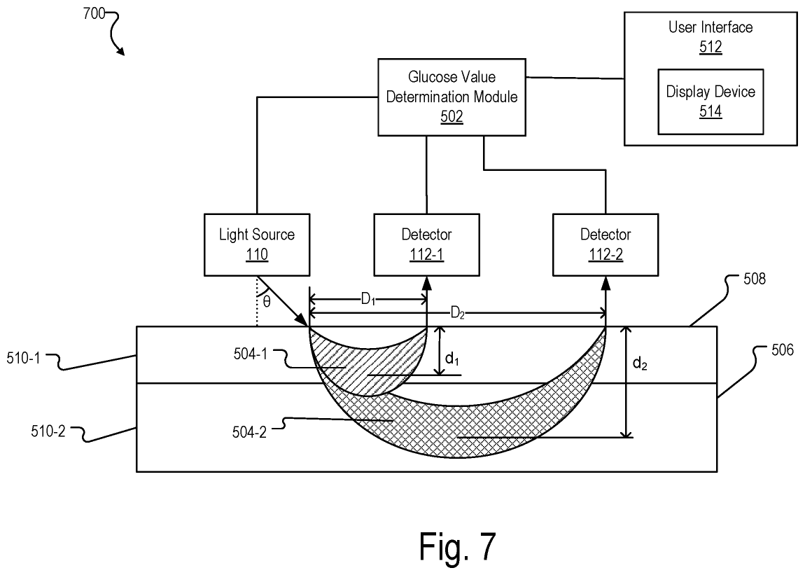

For example, FIG. 7 shows another illustrative implementation 700 including glucose value determination module 502 that may be implemented by a hearing system (e.g., by processor 116) according to the principles described herein and configured to determine the glucose value. As shown, implementation 700 is similar to implementation 500, except that implementation 700 includes a plurality of detectors 112 (e.g., detectors 112-1 to 112-2). Implementation 700 may include additional or alternative components as may serve a particular implementation.

As shown, a first detector 112-1 is positioned at a first distance D₁ (e.g., less than about 10 millimeters, such as about 0 millimeters to about 3 millimeters, such as about 1 millimeter) from light source 110 (e.g., from a location where first light 504-1 contacts surface 508 of tissue 506) such that first detector 112-1 is configured to detect the intensity of first light 504-1. A second detector 112-2 is positioned at a second distance D₂ (e.g., less than about 10 millimeters, such as about 3 millimeters to about 6 millimeters, such as about 4 millimeters to about 5 millimeters) from light source 110 (e.g., from a location where second light 504-2 contacts surface 508 of tissue 506) greater than first distance D₁ such that second detector 112-2 is configured to detect the intensity of second light 504-2. Such a spatial offset between first detector 112-1 and second detector 112-2 may allow second detector 112-2 to detect the intensity of second light 504-2 from greater penetration depths (e.g., second depth d₂).

In some implementations, each of first detector 112-1 and second detector 112-2 may include an optical waveguide extending between detectors 112 and surface 508 of tissue 506 and configured to guide first light 504-1 toward first detector 112-1 and/or second light 504-2 toward second detector 112-2 after being scattered by tissue 506. Additionally or alternatively, each of first detector 112-1 and second detector 112-2 may include a filter (e.g., a notch filter, an edge pass filter, a band pass filter, etc.) configured to filter first light 504-1 at the first excitation frequency after first light 504-1 has been scattered by tissue 506 and/or second light 504-2 at the second excitation frequency after second light 504-2 has been scattered by tissue 506.

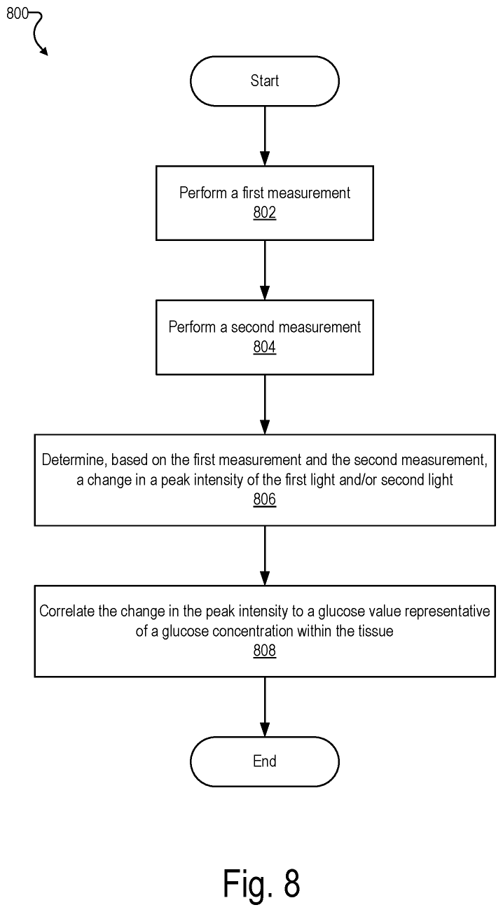

FIG. 8 shows another illustrative method 800 that may be performed by a hearing system according to the principles described herein. While FIG. 8 illustrates exemplary operations according to one embodiment, other embodiments may omit, add to, reorder, and/or modify any of the operations shown in FIG. 8. Moreover, each of the operations depicted in FIG. 8 may be performed in any of the ways described herein. In some implementations, each of the operations depicted in FIG. 8 may be performed by at least one computing device (e.g., by processor 116).

As shown, the hearing system (e.g., glucose value determination module 502) may, at operation 802, perform a first measurement of each of the first light (e.g., first light 504-1) and the second light (e.g., second light 504-2). The first measurement may include causing at least one light source (e.g., light source 110) to emit the first light and the second light toward tissue (e.g., tissue 506) at the ear and causing at least one detector (e.g., detector 112) to detect intensities of the first light and the second light after being scattered by the tissue. For example, the intensities of the first light may be detected at frequencies offset from the first excitation frequency and the intensities of the second light may be detected at frequencies offset from the second excitation frequency.

In some implementations, the first measurement may include determining a pure signal of the measured first light 504-1 and/or the measured second light 504-2, e.g., by applying one or more conversion factors to the intensities of the detected light that was scattered by different layers (e.g., layers 510) of the tissue, such as by determining intensities of the first light that was scattered by tissue at a first depth (e.g., first depth d₁) and intensities of the second light that was scattered by tissue at a second depth (e.g., second depth d₂). The first measurement may be performed at a first time period.

The hearing system may further, at operation 804, perform a second measurement of each of the first light and the second light. The second measurement may include causing the at least one light source to emit the first light and the second light toward tissue at the ear and causing the at least one detector to detect intensities of the first light and the second light after being scattered by the tissue. For example, the intensities of the first light may be detected at frequencies offset from the first excitation frequency and the intensities of the second light may be detected at frequencies offset from the second excitation frequency.

In some implementations, the second measurement may include determining a pure signal of the measured first light 504-1 and/or the measured second light 504-2, e.g., by applying one or more conversion factors to the intensities of the detected light that was scattered by different layers of the tissue, such as by determining intensities of the first light that was scattered by tissue at the first depth and intensities of the second light that was scattered by tissue at the second depth. The second measurement may be performed at a second time period after the first time period (e.g., such as after about 24 hours, after about 12 hours, after about 8 hours, after about 1 hour, after about 30 minutes, etc.).

The hearing system may further, at operation 806, determine based on the first measurement and the second measurement, a change in a peak intensity of the first light and/or the second light, e.g., in the second measurement relative to the first measurement. For example, the first measurement may include identifying a peak intensity representative of a peak of the intensities of the first light at one or more predetermined frequencies offset from the first excitation frequency and/or the intensities of the second light at one or more predetermined frequencies offset from the second excitation frequency. Likewise, the second measurement may include identifying the peak intensity representative of the peak of the intensities of the first light at one or more predetermined frequencies offset from the first excitation frequency and/or the intensities of the second light at one or more predetermined frequencies offset from the second excitation frequency. The change in the peak intensity may be determined, such as by determining a difference between the peak intensity of the first light and/or second light for the first measurement and the peak intensity of the respective first light and/or second light for the second measurement.

The hearing system may further, at operation 808, correlate the change in the peak intensity to a glucose value representative of a glucose concentration within the tissue. In some implementations, the change in the peak intensity may be indicative of a relative change in the glucose concentration within the tissue between the first measurement and the second measurement such that the glucose value may increase as the change in the peak intensity increases and/or decrease as the change in the peak intensity decreases.

In certain embodiments, one or more of the processes described herein may be implemented at least in part as instructions embodied in a non-transitory computer-readable medium and executable by one or more computing devices. In general, a processor (e.g., a microprocessor) receives instructions, from a non-transitory computer-readable medium, (e.g., a memory, etc.), and executes those instructions, thereby performing one or more processes, including one or more of the processes described herein. Such instructions may be stored and/or transmitted using any of a variety of known computer-readable media.

A computer-readable medium (also referred to as a processor-readable medium) includes any non-transitory medium that participates in providing data (e.g., instructions) that may be read by a computer (e.g., by a processor of a computer). Such a medium may take many forms, including, but not limited to, non-volatile media, and/or volatile media. Non-volatile media may include, for example, optical or magnetic disks and other persistent memory. Volatile media may include, for example, dynamic random access memory (“DRAM”), which typically constitutes a main memory. Common forms of computer-readable media include, for example, a disk, hard disk, magnetic tape, any other magnetic medium, a compact disc read-only memory (“CD-ROM”), a digital video disc (“DVD”), any other optical medium, random access memory (“RAM”), programmable read-only memory (“PROM”), electrically erasable programmable read-only memory (“EPROM”), FLASH-EEPROM, any other memory chip or cartridge, or any other tangible medium from which a computer can read.

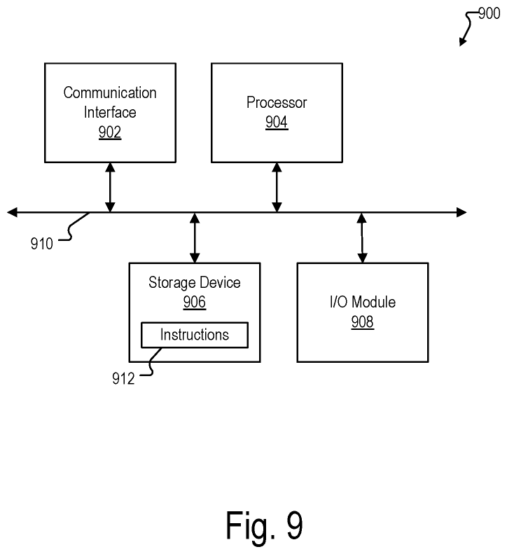

FIG. 9 shows an illustrative computing device 900 that may be specifically configured to perform one or more of the processes described herein. Any of the systems, computing devices, and/or other components described herein may be implemented by computing device 900.

As shown in FIG. 9, computing device 900 may include a communication interface 902, a processor 904, a storage device 906, and an input/output (“I/O”) module 908 communicatively connected one to another via a communication infrastructure 910. While an illustrative computing device 900 is shown in FIG. 9, the components illustrated in FIG. 9 are not intended to be limiting. Additional or alternative components may be used in other embodiments. Components of computing device 900 shown in FIG. 9 will now be described in additional detail.

Communication interface 902 may be configured to communicate with one or more computing devices. Examples of communication interface 902 include, without limitation, a wired network interface (such as a network interface card), a wireless network interface (such as a wireless network interface card), a modem, an audio/video connection, and any other suitable interface.

Processor 904 generally represents any type or form of processing unit capable of processing data and/or interpreting, executing, and/or directing execution of one or more of the instructions, processes, and/or operations described herein. Processor 904 may perform operations by executing computer-executable instructions 912 (e.g., an application, software, code, and/or other executable data instance) stored in storage device 906.

Storage device 906 may include one or more data storage media, devices, or configurations and may employ any type, form, and combination of data storage media and/or device. For example, storage device 906 may include, but is not limited to, any combination of the non-volatile media and/or volatile media described herein. Electronic data, including data described herein, may be temporarily and/or permanently stored in storage device 906. For example, data representative of computer-executable instructions 912 configured to direct processor 904 to perform any of the operations described herein may be stored within storage device 906. In some examples, data may be arranged in one or more databases residing within storage device 906.

I/O module 908 may include one or more I/O modules configured to receive user input and provide user output. I/O module 908 may include any hardware, firmware, software, or combination thereof supportive of input and output capabilities. For example, I/O module 908 may include hardware and/or software for capturing user input, including, but not limited to, a keyboard or keypad, a touchscreen component (e.g., touchscreen display), a receiver (e.g., an RF or infrared receiver), motion sensors, and/or one or more input buttons.

I/O module 908 may include one or more devices for presenting output to a user, including, but not limited to, a graphics engine, a display (e.g., a display screen), one or more output drivers (e.g., display drivers), one or more audio speakers, and one or more audio drivers. In certain embodiments, I/O module 908 is configured to provide graphical data to a display for presentation to a user. The graphical data may be representative of one or more graphical user interfaces and/or any other graphical content as may serve a particular implementation.

In the preceding description, various exemplary embodiments have been described with reference to the accompanying drawings. It will, however, be evident that various modifications and changes may be made thereto, and additional embodiments may be implemented, without departing from the scope of the invention as set forth in the claims that follow. For example, certain features of one embodiment described herein may be combined with or substituted for features of another embodiment described herein. The description and drawings are accordingly to be regarded in an illustrative rather than a restrictive sense.

What is claimed is:

1. A hearing system comprising:
a housing configured to be worn at an ear of a user, the housing comprising:
at least one light source configured to emit a first light and a second light toward tissue at the ear, the first light having a first excitation frequency and the second light having a second excitation frequency different from the first excitation frequency; and
at least one detector configured to detect intensities of the first light at frequencies offset from the first excitation frequency after the first light has been scattered by the tissue and intensities of the second light at frequencies offset from the second excitation frequency after the second light has been scattered by the tissue; and
a processing unit configured to determine, based on the intensities of the first light and the second light, a glucose value representative of a glucose concentration within the tissue.

2. The hearing system of claim 1, wherein the housing comprises:
a microphone configured to detect an audio signal presented to the user; and
an output transducer configured to output the audio signal detected by the microphone to the user.

3. The hearing system of claim 1, wherein the at least one detector is configured to detect the intensities of the first light and the second light at a single location relative the at least one light source.

4. The hearing system of claim 1, wherein the at least one detector includes:
a first detector configured to detect the intensities of the first light and positioned at a first distance from the at least one light source; and
a second detector configured to detect the intensities of the second light and positioned at a second distance from the at least one light source greater than the first distance.

5. The hearing system of claim 1, wherein a wavelength of the second light is greater than a wavelength of the first light.

6. The hearing system of claim 1, wherein the first light penetrates the tissue at a first depth and the second light penetrates the tissue at a second depth greater than the first depth such that the second light penetrates deeper into the tissue than the first light.

7. The hearing system of claim 6, wherein the determining the glucose value includes determining the intensities of the first light that was scattered by the tissue at the first depth and determining the intensities of the second light that was scattered by the tissue at the second depth.

8. The hearing system of claim 1, wherein the determining the glucose value includes identifying one or both of the intensities of the first light at one or more predetermined frequencies offset from the first excitation frequency or the intensities of the second light at one or more predetermined frequencies offset from the second excitation frequency.

9. The hearing system of claim 1, wherein the determining the glucose value includes applying one or more conversion factors to the detected intensities.

10. The hearing system of claim 1, wherein the determining the glucose value includes:
performing a first measurement of each of the first light and the second light;
performing a second measurement of each of the first light and the second light;
determining, based on the first measurement and the second measurement, a change in a peak intensity of one or both of the first light or the second light; and
correlating the change in the peak intensity to the glucose value.

11. The hearing system of claim 10, wherein the change in the peak intensity is indicative of a change in the glucose concentration within the tissue between the first measurement and the second measurement.

12. The hearing system of claim 10, wherein the glucose value increases as the change in the peak intensity increases and wherein the glucose value decreases as the change in the peak intensity decreases.

13. The hearing system of claim 10, wherein each of the first measurement and the second measurement includes:
determining the intensities of the first light that was scattered by the tissue at a first depth and determining the intensities of the second light that was scattered by the tissue at a second depth; and
identifying the peak intensity of one or both of the intensities of the first light at one or more predetermined frequencies offset from the first excitation frequency or the intensities of the second light at one or more predetermined frequencies offset from the second excitation frequency.

14. The hearing system of claim 1, wherein the first light and the second light each include a monochromatic infrared light.

15. The hearing system of claim 1, wherein the at least one detector includes a filter configured to filter the first light at the first excitation frequency after the first light has been scattered by the tissue and the second light at the second excitation frequency after the second light has been scattered by the tissue.

16. The hearing system of claim 1, wherein the processing unit is further configured to provide the glucose value for display by a display device.

17. A method comprising:
causing, by at least one computing device, at least one light source to emit a first light and a second light toward tissue at an ear of a user, the first light having a first excitation frequency and the second light having a second excitation frequency different from the first excitation frequency;
causing, by the at least one computing device, at least one detector to detect intensities of the first light at frequencies offset from the first excitation frequency after the first light has been scattered by the tissue and intensities of the second light at frequencies offset from the second excitation frequency after the second light has been scattered by the tissue; and
determining, by the at least one computing device and based on the intensities of the first light and the second light, a glucose value representative of a glucose concentration within the tissue.

18. The method of claim 17, wherein the determining the glucose value includes:
performing a first measurement of each of the first light and the second light;
performing a second measurement of each of the first light and the second light;
determining, based on the first measurement and the second measurement, a change in a peak intensity of one or both of the first light or the second light; and
correlating the change in the peak intensity to the glucose value.

19. The method of claim 18, wherein each of the first measurement and the second measurement includes determining the intensities of the first light that was scattered by the tissue at a first depth and determining the intensities of the second light that was scattered by the tissue at a second depth greater than the first depth.

20. A hearing device configured to be worn at an ear of a user, the hearing device comprising:
a microphone configured to detect an audio signal presented to the user;
an output transducer configured to output the audio signal detected by the microphone to the user;
at least one light source configured to emit a first light and a second light toward tissue at the ear, the first light having a first excitation frequency and the second light having a second excitation frequency different from the first excitation frequency; and
at least one detector configured to detect intensities of the first light at frequencies offset from the first excitation frequency after the first light has been scattered by the tissue and intensities of the second light at frequencies offset from the second excitation frequency after the second light has been scattered by the tissue for determining, based on the intensities of the first light and the second light, a glucose value representative of a glucose concentration within the tissue.
